(19) United States
(12) Patent Application Publication
(10) Pub. No.: US 2023/0181662 A1
(43) Pub. Date: Jun. 15, 2023

(54) USES OF ANTRODIA CINNAMOMEA EXTRACT IN MANUFACTURING PRODUCTS FOR REDUCING EXPRESSION AND TREATING ASSOCIATED DISEASES OF ANGIOTENSIN CONVERTING ENZYME 2

(71) Applicant: ALPS BIOTECH CO., LTD., Taipei City (TW)

(72) Inventors: Shui-Tein Chen, Taipei City (TW); Kuo-Feng Hua, Taipei City (TW)

(73) Assignee: ALPS BIOTECH CO., LTD., Taipei City (TW)

(21) Appl. No.: 17/997,041

(22) PCT Filed: Apr. 27, 2021
(86) PCT No.: PCT/CN2021/090292

§ 371 Date: Oct. 25, 2022

Related U.S. Application Data
(60) Provisional application No. 63/016,665, filed on Apr. 28, 2020.

(51) Int. Cl. A61K 36/07; A61P 31/14; A61P 9/00; A23L 33/145

(52) U.S. Cl. CPC A61K 36/07; A61P 31/14; A61P 9/00; A23L 33/145; A61K 2236/333

(57) ABSTRACT

Disclosed herein are a method of treating, reducing the risk of, preventing, or alleviating angiotensin converting enzyme 2 (ACE-2) associated state in a subject, comprising administering to the subject a therapeutically effective amount of Antrodia cinnamomea extract (Ant-Ex) or fraction 3 thereof (AE-F03). Also provided herein are uses of Ant-Ex or AE-F03 in reducing ACE-2 expression and manufacturing a drug or a food supplement for treating, reducing the risk of, preventing, or alleviating ACE-2 associated state.

### CROSS-REFERENCE TO RELATED APPLICATION

This application claims the benefit of U.S. Provisional Application No. 63/016,665, filed Apr. 28, 2020.

### BACKGROUND OF INVENTION

Antrodia cinnamomea (synonym Taiwanofungus camphoratus) is an endemic polyporus medicinal mushroom with orange/red fruiting bodies. It grows only on the inner cavities of the endemic tree species Cinnamomum kanehirae hayata, which belongs to Lauraceae family. The mushroom has commonly been used in ethnomedicine as a remedy for cancer, hypertension, hangover, and food and drug intoxication.

Previous scientific reports indicated that the Antrodia cinnamomea extracts and purified compounds have numerous biological activities including anti-cancer, anti-inflammatory, hepatoprotective, anti-oxidant, neuroprotective and immune-modulatory, and so on. The extracts of Antrodia cinnamomea contains various metabolites including terpenoids, benzenoids, lignans, succinic and maleic acid derivatives, and polysaccharides. Among these, triterpenoids are major components in fruiting bodies, and have received more attention due to their potent anticancer, anti-inflammatory, immunemodulatory, and anti-diabetic properties.

The angiotensin-converting enzyme 2 (ACE-2) is a monocarboxypeptidase known for cleaving several peptides within the renin-angiotensin system and other substrates, such as apelin. This enzyme is barely present in the circulation, but widely expressed in organs, such as the kidneys and the gastrointestinal tract, with relatively low level of expression in lungs.

### SUMMARY OF INVENTION

The present disclosure, at least in part, is based on the development of Antrodia cinnamomea extract (Ant-Ex) or fraction 3 thereof (AE-F03), which showed potent activities in reducing angiotensin converting enzyme 2 (ACE-2) expression so as to treat, reduce the risk of, prevent, or alleviate ACE-2 associated state.

Accordingly, one aspect of the present disclosure relates to a method of treating, reducing the risk of, preventing, or alleviating angiotensin converting enzyme 2 (ACE-2) associated state in a subject, comprising administering to the subject a therapeutically effective amount of Antrodia cinnamomea extract (Ant-Ex) or fraction 3 thereof (AE-F03).

In another aspect, provided herein is a use of Antrodia cinnamomea extract (Ant-Ex) or fraction 3 thereof (AE-F03) in manufacturing a drug or a food supplement for treating, reducing the risk of, preventing, or alleviating angiotensin converting enzyme 2 (ACE-2) associated state.

Further, the present disclosure provides a use of Antrodia cinnamomea extract (Ant-Ex) or fraction 3 thereof (AE-F03) in reducing angiotensin converting enzyme 2 (ACE-2) expression.

In some embodiments, the subject is mammalian, preferably a human.

The ACE-2 associated state disclosed herein may be a blood pressure related disease or disorder. Preferably, said ACE-2 associated state is selected from the group consisting of: chronic heart failure, left ventricular hypertrophy, acute heart failure, and cardiomyopathy. Said ACE-2 associated state may also be congestive heart failure, arterial hypertension or myocardial infarction

The ACE-2 associated state disclosed herein may be cell proliferation disorder. Exemplary cell proliferation disorder includes, but are not limited to, smooth cell proliferation disorder, preferably vascular stenosis.

The ACE-2 associated state disclosed herein may be kidney disease or disorder, or kinetensin associated disorder. Exemplary kinetensin associated disorder includes, but are not limited to, abnormal vascular permeability, local and systemic allergic reactions, eczema, asthma, and anaphylactic shock.

The ACE-2 associated state disclosed herein may be inflammation. Exemplary inflammation includes, but are not limited to, systemic inflammatory response syndromes (SIRS), polytrauma, inflammatory bowel disease, acute and chronic pain, bone destruction in rheumatoid and osteo arthritis, periodontal disease, dysmeorrhea, premature labor, brain edema following focal injury, diffuse axonal injury, allergic disorders, wound healing, or scar formation.

The ACE-2 associated state disclosed herein may be virus infection, preferably corona virus infection. Exemplary corona virus infection includes, but are not limited to, severe acute respiratory syndrome coronavirus (SARS-CoV) infection, Middle East respiratory syndrome coronavirus (MERS-CoV) infection or severe acute respiratory syndrome coronavirus 2 (SARS-CoV-2) infection.

In any of the methods or uses disclosed herein, the Antrodia cinnamomea extract is obtained by: extracting dried fruiting bodies of Antrodia cinnamomea via 95% (v/v) ethanol and comprises a fraction 3 thereof (AE-F03).

In any of the methods disclosed herein, the subject has undergone or is undergoing an additional treatment of the disease.

The details of one or more embodiments of the invention are set forth in the description below. Other features or advantages of the present invention will be apparent from the following drawing and detailed description of several embodiments, and also from the appended claims.

### BRIEF DESCRIPTION OF DRAWINGS

FIG. 1 shows that Antrodia cinnamomea extract (Ant-Ex) reduces angiotensin converting enzyme 2 (ACE-2) protein expression. Human lung cancer CL1-5 cells were incubated with 50 μg/mL Ant-Ex for 24 hours, ACE-2 protein expression levels were analyzed by Western blot. ACE2 antibody was purchased from Proteintech (catalog number: 21115-1-AP).

FIG. 2 shows that Ant-Ex reduces ACE-2 protein expression in a time dependent manner. Human lung cancer CL1-5 cells were incubated with 50 μg/mL Ant-Ex for 0-24 hours, ACE-2 protein expression levels were analyzed by Western blot. ACE-2 antibody was purchased from Proteintech (catalog number: 21115-1-AP).

FIG. 3 shows that Ant-Ex reduces ACE-2 protein expression in a dose dependent manner. Human lung cancer CL1-5 cells were incubated with 12.5-50 μg/mL Ant-Ex for 24 hours, ACE-2 protein expression levels were analyzed by Western blot. ACE-2 antibody was purchased from Proteintech (catalog number: 21115-1-AP).

FIG. 4 shows that Ant-Ex reduces ACE-2 protein expression in a dose dependent manner. Human lung cancer CL1-0 cells were incubated with 12.5-50 μg/mL Ant-Ex for 24 hours, ACE-2 protein band is indicated by arrow. GAPDH from the same cells were also detected as protein-quantity control.

FIG. 5 shows that Ant-Ex reduces ACE-2 mRNA expression. Human lung cancer CL1-5 cells were incubated with 50 μg/mL Ant-Ex or vehicle for 1, 2 or 3 hours, the ACE-2 mRNA expression levels were analyzed by RT-qPCR.

FIG. 6 shows the inhibition of enzyme activity of ACE-2 by Ant-Ex. (A) The principle for ACE-2 activity assay. (B) Ant-Ex (1, 5, 10 mg/mL) can reduce enzyme activity (about 40%) and the inhibition is shown by column chart. (C) Various concentration of Ant-Ex (40 μM, 200 μM and 400 μM) is subjected into ACE-2 enzyme activity assay, and the result presents the Ki of Ant-Ex is about 590.9 μM.

FIG. 7 shows the inhibition of enzyme activity of ACE-2 by various constituents in Ant-Ex with the assay system as mentioned in FIG. 7(A). The tested constituents were Antcin A, Antcin B, Antcin C, Antcin H, Antcin K, DSA (dehydrosulphurenic acid) and DEA (dehydroeburicoic acid). Each ingredient with 20 μg/mL was assayed and their inhibition ability was shown in column chart. Among these, except for Antcin B, all of these tested compounds had inhibition ability on ACE-2 enzymatic activity. Among the tested compounds, DSA, Antcin A and Antcin K were shown to have stronger inhibition activity.

FIG. 8 shows the cellular inhibition assay of the interaction of SARS-CoV-2 spike protein (S protein) and ACE-2 receptor by Ant-Ex. (A) The principle and flow-chart of this cell-assay system. The system contains a pseudovirus with SARS-CoV-2 S protein and carrying a luciferase reporter gene and 293T cells which can overexpress ACE-2 protein on the cell surface. (B) Ant-Ex (0-50 μg/mL) can inhibit the interaction of spike protein and ACE-2 receptor with about 80% inhibition at 50 μg/mL (p<0.05).

FIG. 9 shows the inhibition assay of SARS-CoV-2 infecting Vero-E6 by Ant-Ex. (A) Vero-E6 cells were pretreated with the Ant-Ex at the indicated concentration for 1 hour at 37° C. and then were adsorbed with severe acute respiratory syndrome coronavirus 2 (SARS-CoV-2) at 100 PFU (MOI=0.01) for 1 hour at 37° C. The virus was removed and cells were added with fresh medium with the compound at the indicated concentration for 1 day incubation. Cells were fixed and immunostained with anti-SARS-CoV-2 N protein antibody plus anti-human IgG-488. For cell viability test, the Vero-E6 cells were treated with the Ant-Ex at the indicated concentration for 1 day at 37° C. The cell viability was determined by Cell Counting Kit-8 (CCK-8). The fluorescent signal was quantified by high-content imaging and the infection rate of no compound treatment was set as 100%. The IC50 and CC50 were calculated by Prism software. (B) For ACE-2 expression test, Vero-E6 cells were treated with the compound at the indicated concentration for 24 hours at 37° C. Cells were fixed and immunostained with anti-hACE-2 antibody (GTX01160) plus anti-rabbit IgG-568. The fluorescent signal was quantified by high-content imaging and expression level of no compound treatment was set as 100%.

FIG. 10 shows that (A) AE-F03 reduces ACE-2 protein expression (about 36%) and have no significant cell toxicity. Human lung cancer CL1-5 cells were incubated with 12.5-50 μg/mL AE-F03 or without AE-F03 for 24 hours. GAPDH from the same cells were also detected as protein-quantity control. (B) AE-F03 inhibits the enzyme activity of ACE-2 protein. ACE-2 protein were treated with 100 μg/mL or without AE-F03 for 30 min then treated with substrate containing fluorescence making group for another 10 min. If the inhibition exists, the fluorescence strength decreases. The principal of this enzyme activity assay is mentioned as FIG. 7(A). After AE-F03 treatment, the enzyme activity of ACE-2 is inhibited by about 10%. (C) The major constituents of AE-F03, Antcin K, DSA and DEA, also have ability of inhibition on ACE-2 by performing inhibition assay (with 20 μg/mL each compound or without as control). DSA is the strongest inhibitor with more than 30% inhibition. (D) Vero-E6 cells were pretreated with AE-F03 at the indicated concentration for 1 hour at 37° C. and then were adsorbed with SARS-CoV-2 at 100 PFU (MOI=0.01) for 1 hour at 37° C. The virus was removed and cells were added with fresh medium with the compound at the indicated concentration for 1 day incubation. Cells were fixed and immunostained with anti-SARS-CoV-2 N protein antibody plus anti-human IgG-488. For cell viability test, the Vero-E6 cells were treated with the compound at the indicated concentration for 1 day at 37° C. The cell viability was determined by Cell Counting Kit-8 (CCK-8). The fluorescent signal was quantified by high-content imaging and the infection rate of no compound treatment was set as 100%. The IC50 and CC50 were calculated by Prism software.

FIG. 11 shows (A) the HPLC chromatography of Ant-Ex and (B) the retention time (RT), area, high and the name of the compounds within Ant-Ex.

FIG. 12 shows that the flow chart of fractioning AE-F03 from Ant-Ex.

FIG. 13 shows (A) the HPLC chromatography of AE-F03 and (B) the retention time (RT), area, high and the name of the compounds within AE-F03.

### DETAILED DESCRIPTION OF INVENTION

The present application pertains to a method of treating, reducing the risk of, preventing, or alleviating angiotensin converting enzyme 2 (ACE-2) associated state in a subject, comprising administering to the subject a therapeutically effective amount of Antrodia cinnamomea extract (Ant-Ex) or fraction 3 thereof (AE-F03), such that the ACE-2 associated state is treated. Wherein, the AE-F03 comprise Antcin K, DSA, DEA and 3β,15α-Dihydroxylanosta-7,9(11),24-trien-21-oic acid (DLTO). In one embodiment, the AE-F03 may be extracted and fractioned from Antrodia cinnamomea or any other known origins. Alternatively, the AE-F03 may be a composition mixing at least the components mentioned above. In one embodiment, Ant-Ex and/or AE-F03 described herein are useful for treating ACE-2 mediated disorders, which, as used herein, refer to any medical condition associated with increased levels of ACE-2 or increased sensitivity to ACE-2.

The language “ACE-2 associated state” includes those states which are associated with ACE-2, ACE-2 substrates, or the products of its metabolic pathways. In one embodiment, ACE-2 associated state includes disorders which are characterized by aberrant levels of ACE-2 activity, levels of ACE-2 substrate and/or ACE-2 metabolic products. ACE-2 associated states may include, for example, high blood pressure, high blood pressure related diseases and disorders, high susceptibility to virus infection, e.g. corona virus, and/or arterial hypertension.

In an embodiment, high blood pressure related diseases and disorders may include, e.g., congestive heart failure (CHF), chronic heart failure, left ventricular hypertrophy, acute heart failure, myocardial infarction, and/or cardiomyopathy.

In an embodiment, ACE-2 associated states also include dysregulated cell proliferation, such as smooth cell proliferation. Smooth muscle cell proliferation in the intima of muscular arteries is a primary cause of vascular stenosis in atherosclerosis, after vascular surgery, and after coronary angioplasty. Several animal studies have indicated that the renin-angiotensin system plays an important role in this vascular response to injury.

In an embodiment, ACE-2 associated states include kidney diseases or disorders, e.g., renal failure. Thus, based at least in part on the fact that ACE-2 is expressed in kidney and is homologous to angiotensin converting enzyme, ACE-2 modulating compounds may be used for treating and preventing renal diseases or disorders, either alone or in combination with known angiotensin converting enzyme inhibitors.

In an embodiment, ACE-2 associated states include hyperadrenergic states, such as acute myocardial infarction (AMI) and some ventricular arrhythmias. ACE-2 is known to cleave the C-terminal amino acid (leucine) from kinetensin. Kinetensin is a nine amino acid peptide having SEQ ID NO: 23 (see U.S. Ser. No. 09/163,648) which has been reported to induce a dose-dependent release of histamine from mast cells, as well as induce a dose-dependent increase in vascular permeability when injected intradermally into rats. Accordingly, modulating the plasma and/or tissue level of kinetensin, such as by modulating the hydrolysis of the C-terminal amino acid from kinetensin, should be useful for treating conditions that are caused by, or contributed to by, an abnormal kinetensin level. Such conditions include those caused by, or contributed to by, an abnormal histamine release from mast cells and/or by an abnormal vascular permeability. Since excessive histamine release is associated with local or systemic allergic reactions, including eczema, asthma, anaphylactic shock, these conditions are included in the “ACE-2 associated states.”

In an embodiment, ACE-2 associated states include, for example, systemic inflammatory response syndromes (SIRS), sepsis, polytrauma, inflammatory bowel disease, acute and chronic pain, bone destruction in rheumatoid and osteo arthritis and periodontal disease, dysmenorrhea, premature labor, brain edema following focal injury, diffuse axonal injury, stroke, reperfusion injury and cerebral vasospasm after subarachnoid hemorrhage, allergic disorders including asthma, adult respiratory distress syndrome, wound healing and scar formation.

The term “treating” includes curing as well as ameliorating at least one symptom of the state, disease or disorder.

The term “alleviating” does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

The term “administering” includes routes of administration which allow the Ant-Ex or AE-F03 to perform its intended function, e.g. reducing expression of ACE-2, inhibiting the function of ACE-2 and/or treating an ACE-2 associated state. Examples of routes of administration include parenteral (e.g., subcutaneous injection, intravenous injection, and intramuscular injection), intraperitoneal injection, enteral, inhalation, transdermal or the like, depending on the states being treated, e.g. the severity of the disease or infection to be treated. In an embodiment, the injection may be bolus injections or continuous infusion.

The Ant-Ex or AE-F03 may be administered using any amount and any route of administration effective for attenuating infectivity of the viruses, e.g. severe acute respiratory syndrome coronavirus (SARS-CoV), Middle East respiratory syndrome coronavirus (MERS-CoV) or severe acute respiratory syndrome coronavirus 2 (SARS-CoV-2) or any other corona virus strains. Thus, the term “effective amount” used herein refers to a nontoxic but sufficient amount of the antiviral agent to provide the desired treatment of viral infection. The exact amount required will vary from subject to subject, depending on the species, age, and general condition of the subject, the severity of the infection, the particular antiviral agent and its mode of administration, and the like.

Although the Ant-Ex or AE-F03 can be administered to any patient who is susceptible to virus infection, preferably, corona virus infection, the Ant-Ex or AE-F03 is intended for the treatment of mammalian hosts, and especially humans.

In view of the inhibitory effect produced by the Ant-Ex or AE-F03, it is anticipated that these composition will be useful not only for therapeutic treatment of infection, but for prophylaxis, as well. The above-noted dosages will be essentially the same whether for treatment or prophylaxis of virus infection, preferably, corona virus infection.

Depending on the route of administration, the Ant-Ex or AE-F03 can be coated with or disposed in a selected material to protect it from natural conditions which may detrimentally affect its ability to perform its intended function. The Ant-Ex or AE-F03 can be administered alone or with a pharmaceutically acceptable carrier. Further, the AE-F03 can be administered as a mixture, which also can be co-administered with a pharmaceutically acceptable carrier. The Ant-Ex or AE-F03 can be administered prior to the onset of an ACE-2 mediated state, or after the onset of an ACE-2 mediated state. The Ant-Ex or AE-F03 can also be administered as a prodrug which is converted to another form in vivo.

The compounds are preferably formulated in dosage unit form for ease of administration and uniformity of dosage. Dosage unit form as used herein refers to a physically discrete unit of agent appropriate for the patient to be treated. Each dosage should contain the quantity of active material calculated to produce the desired therapeutic effect either as such, or in association with the selected pharmaceutically acceptable carrier. The Ant-Ex or AE-F03 may be prepared in various forms for administration, including tablets, caplets, pills or dragees, or can be filled in suitable containers, such as capsules, or, in the case of suspensions, filled into bottles.

As used herein, “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. In other word, “pharmaceutically acceptable carrier” as used herein means a pharmaceutically acceptable material, composition or vehicle, such as a liquid or solid filler, diluent, excipient, solvent or encapsulating material, involved in carrying or transporting the Ant-Ex or AE-F03 within or to the subject such that it can performs its intended function. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof.

Except insofar as any conventional carrier medium is incompatible with the Ant-Ex or AE-F03, such as by producing any undesirable biological effect or otherwise interacting in a deleterious manner with any other component(s) of the Ant-Ex or AE-F03, its use is contemplated to be within the scope of this disclosure. In the pharmaceutical compositions of the invention, the active agent may be present in an amount of at least 0.5% and not more than 99% by weight based on the total weight of the composition, including carrier medium and/or auxiliary agent(s). Preferably, the proportion of active agent, e.g. Ant-Ex or AE-F03, varies between 5%-70% by weight of the composition. Pharmaceutical organic or inorganic solid or liquid carrier media suitable for enteral or parenteral administration can be used to make up the composition. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

The language “therapeutically effective amount” of the compound is that amount necessary or sufficient to treat, reduce the risk of, prevent, or alleviate an ACE-2 associated state, e.g. prevent the various morphological and somatic symptoms of an ACE-2 associated state. The effective amount can vary depending on such factors as the size and weight of the subject, the type of illness, or the purity of the Ant-Ex or AE-F03. One of ordinary skill in the art would be able to study the aforementioned factors and make the determination regarding the effective amount of the Ant-Ex or AE-F03 without undue experimentation.

The effective amount can be determined through consideration of the toxicity and therapeutic efficacy of the Ant-Ex or AE-F03 by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to non-affected cells, e.g. uninfected cells, and thereby reducing side effects.

Generally, for administration of any of the Ant-Ex or AE-F03 described herein, an initial candidate dosage can be about 50-250 mg/kg. For the purpose of the present disclosure, a typical daily, weekly, every two weeks, or every three weeks dosage might range from about any of 50 mg/kg to 60 mg/kg to 70 mg/kg to 80 mg/kg to 90 mg/kg to 100 mg/kg, 120 mg/kg, 150 mg/kg, to 180 mg/kg, to 200 mg/kg to 250 mg/kg or more, depending on the factors mentioned above. For repeated administrations over several days, weeks, months, or longer, depending on the condition, the treatment is sustained until a desired suppression of symptoms occurs or until sufficient therapeutic levels are achieved to alleviate a target disease or disorder, or a symptom thereof. In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. An exemplary dosing regimen comprises administering an initial dose of about 100 mg/kg every 3 weeks, followed by a maintenance dose of about 50 mg/kg once in 6 weeks, or followed by a maintenance dose of about 80 mg/kg every 3 weeks. However, other dosage regimens may be useful, depending on the pattern of pharmacokinetic decay that the practitioner wishes to achieve. For example, dosing of 80 mg/kg once in every 3 weeks in combination treatment is contemplated. The progress of this therapy is easily monitored by conventional techniques and assays. The dosing regimen can vary over time.

In some embodiments, for an adult patient of normal weight, doses ranging from about 0.1 to 5.0 mg/kg may be administered. In some examples, the dosage of the Ant-Ex or AE-F03 described herein can be 50-250 mg/kg. The particular dosage regimen, i.e., dose, timing and repetition, will depend on the particular individual and that individual's medical history, as well as the properties of the individual agents (such as the half-life of the agent, and other considerations well known in the art).

General Techniques

The practice of the present invention will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are provided to describe the invention in further detail. The examples below are non-limiting and are merely representative of various aspects and features of the present invention.

ACE-2 in its full-length form is a membrane-bound enzyme (˜98%), whereas its shorter (soluble) form (˜2%) circulates in blood at very low levels (Renhong et al., 2020). Measuring the membrane-bound ACE-2 in vivo is technically challenging, most publications from humans reports the levels of ACE-2 activity in blood that reflect the soluble ACE-2 protein circulating at very low levels (Ramchand et al., 2020). Several observations and experimental evidences indicates that beneficial protective role of enhanced ACE-2 in various pathological conditions such as myocardial infarction, atherosclerosis, renal diseases, liver cirrhosis, diabetes, and inflammatory lung injuries (Tikellis and Thomas, 2012).

However, previous studies of coronaviruses that cause SARS have reveal that they bind to ACE-2 in alveoli pulmonis through their surface spike proteins and then cause lung damage and even lung function failure (Kuba et al., 2005). Recent studies also confirmed that severe acute respiratory syndrome coronavirus 2 (SARS-CoV-2) entry depends on membrane-bound (full-length) ACE-2, which act as a receptor for the SARS-CoV-2 spike protein. Binding of the spike protein to ACE-2, along with proteolytic cleavage of ACE-2 by transmembrane serine protease 2 (TMPRSS2), facilitate entry of the virus into cells, viral replication and cell-to-cell transmission. Consistently, in the cell-cell fusion system, SARS-CoV-2 protein could effectively mediate the formation of syncytium between the effector cells and the target cells in the absence of an exogenous proteolytic enzyme, e.g., trypsin, while SARS-CoV-2 S protein could not (Shuai Xia et al., 2020). It is known that the expression and distribution of ACE-2 is high in oral mucosa epithelial, type II alveolar (AT2) of lung, stratified epithelial, enterocytes from ileum and colon, cholangiocytes, myocardial, kidney proximal tubule, and bladder urothelial cells. These reports indicate that those organs with high ACE-2-expressing cells might be potential target for SARS-CoV-2 infection.

In normal human lung tissues, ACE-2 is express in type I and II alveolar epithelial cells. Further, the expression of viral receptor ACE-2 is concentrated in a small number of type II alveolar (AT2) cells, and makes these AT2 cells are likely to be the target cells of SARS-CoV-2. It is interesting to note that 0.64% of human lung cells express ACE-2, and more than 80% of total ACE-2 expression is found in AT2 cells.

### Example 1: Extraction and Fraction of Antrodia cinnamomea

(i) Extraction of Antrodia cinnamomea (Ant-Ex)

Method of obtaining Ant-Ex: The fruiting bodies of A. cinnamomea are identified by Prof. Shui-Tein Chen at Institute of Biological Chemistry, Academia Sinica, Taiwan. Dried (45° C., 48 hours) and grounded powder of the fruiting bodies (50 g) of A. cinnamomea was extracted with both 2-20 volumes (v/w) of 95% alcohol, 37° C. (stirring 7 hours); and water, 100° C. (stirring 5 hours). The extracted solution were then combined together as the extract.

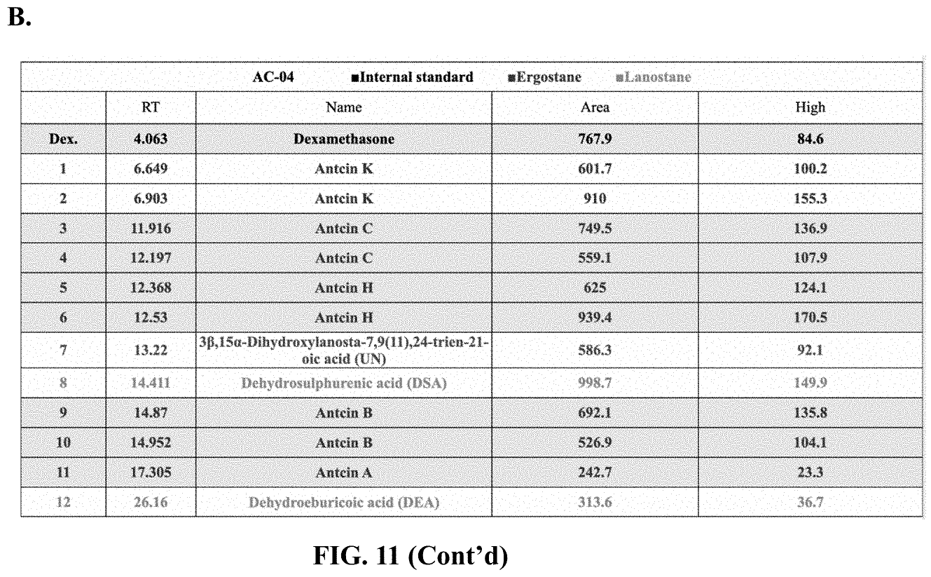

The extract was decanted and filtered through Whatman No. 1 paper, and the solvent was removed under reduced pressure using a rotary evaporator at 45° C. The product was finally lyophilized to get the crude extract Ant-Ex powder (16 g, 32%, w/w). In order to isolate the compounds responsible for the observed ACE-2 inhibition, preliminary HPLC analysis of Ant-Ex revealed the presence of some known compounds in the extract (FIG. 11). Then, the Ant-Ex was subjected to be further fractioned with different solvents, in which AE-F03 as the major fraction.

(ii) Fraction of Antrodia cinnamomea (AE-F03)

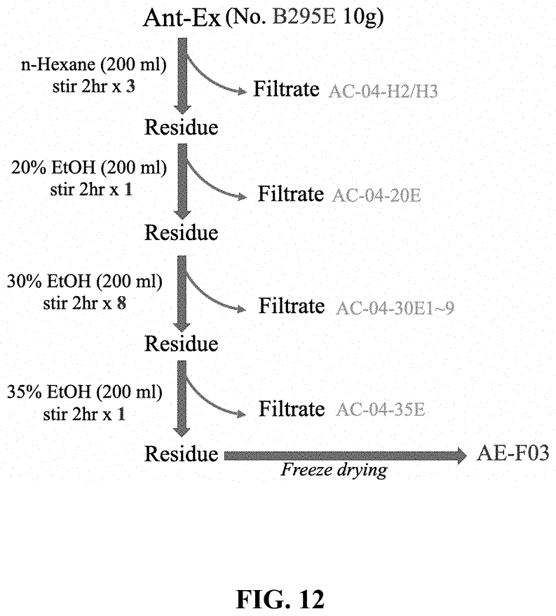

Method of obtaining AE-F03: From the above extract of A. cinnamomea (Ant-Ex), the sample was first washed with n-Hexane 200 mL, stirring 2 hours, 3 times, and kept the residue. Then, the sample was sequentially washed with 20% Ethanol, 1 time (stirring 2 hours); 30% Ethanol, 8 times; and 35% Ethanol, 1 time, separately. The washed residue was kept. (FIG. 12)

The extract was further decanted and filtered through Whatman No. 1 paper, and the solvent was removed under reduced pressure using a rotary evaporator at 45° C. The product was finally lyophilized to get the AE-F03 powder.

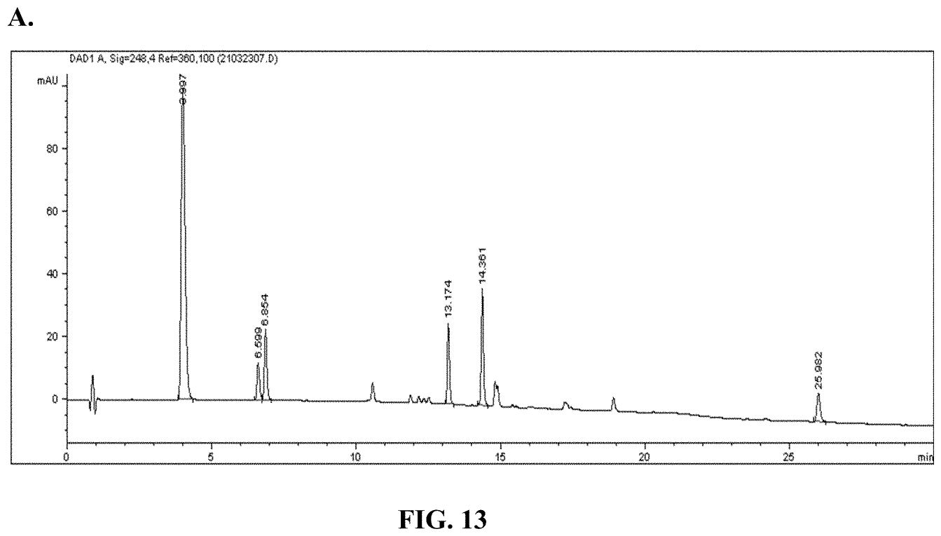

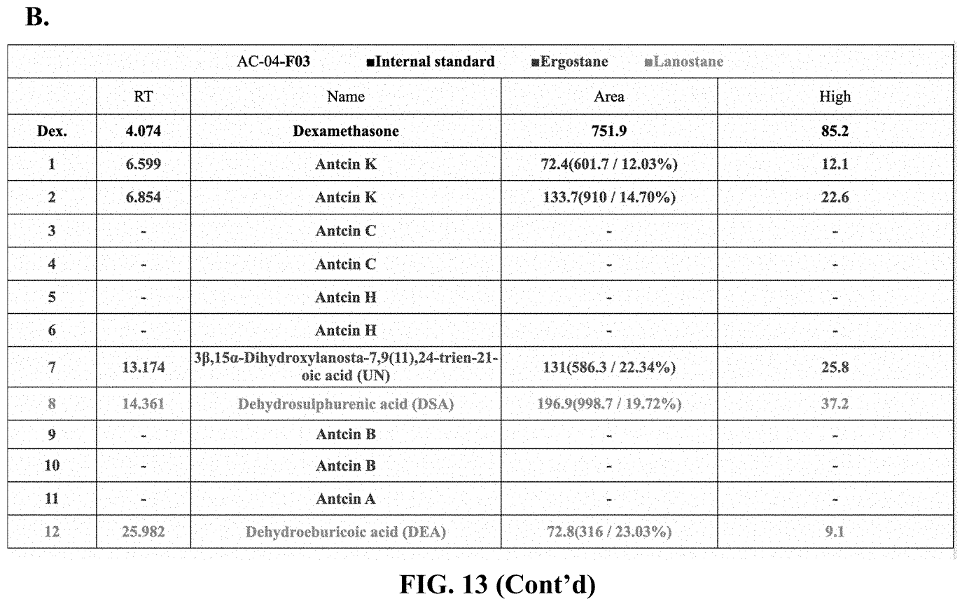

The content of AE-F03 was determined via the HPLC (FIG. 13). The retention time of the peaks appeared to be consistent with the retention time of standard compounds with identical multiplicity as established.

### Example 2: Antrodia cinnamomea Extract (Ant-Ex) Reduces Angiotensin Converting Enzyme 2 (ACE-2) Protein and mRNA Expression

(i) Ant-Ex Reduces ACE-2 Protein Expression

Human lung cancer CL1-5 and CL1-0 cells were incubated with 12.5-50 μg/mL Ant-Ex for 0-24 h. ACE-2 protein expression levels were analyzed by Western blot. ACE-2 antibody was purchased from Proteintech (catalog number: 21115-1-AP). Actin and GAPDH from the same cells were also detected as protein-quantity control.

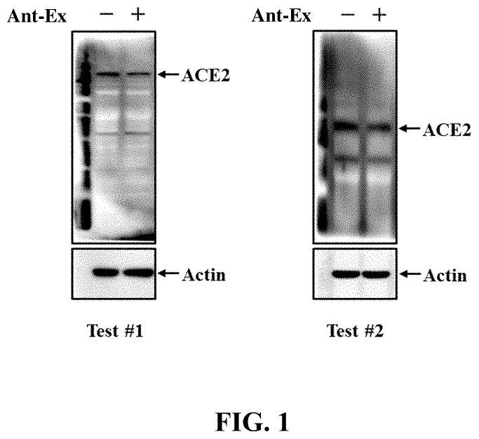

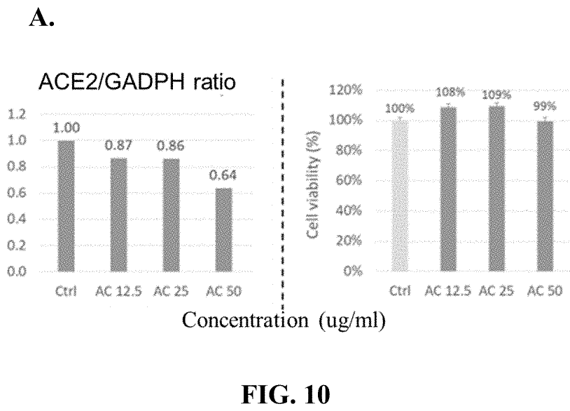

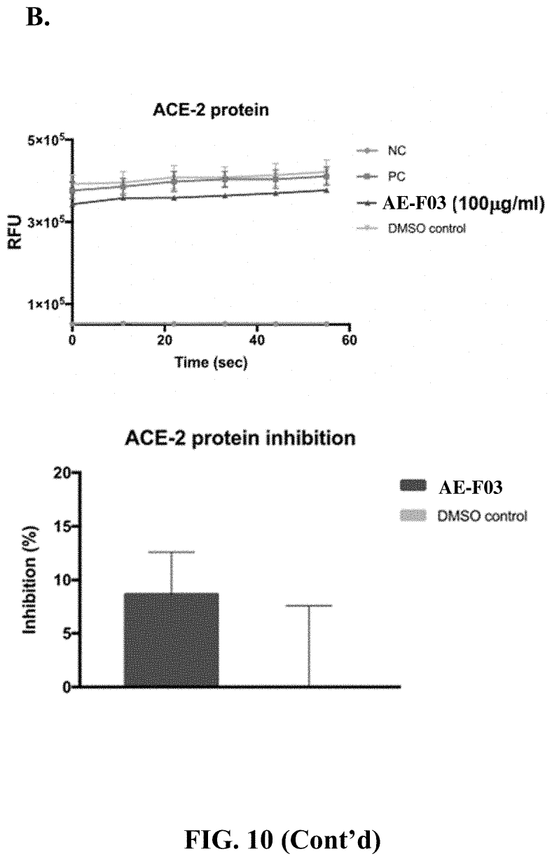

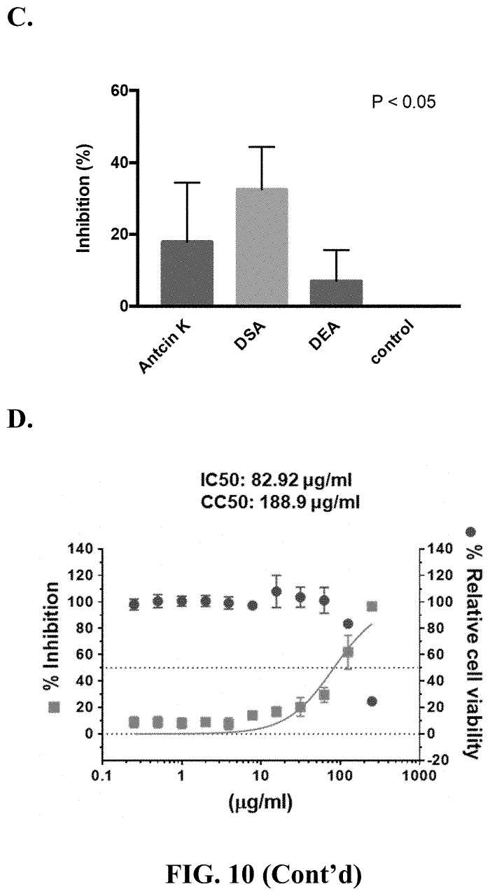

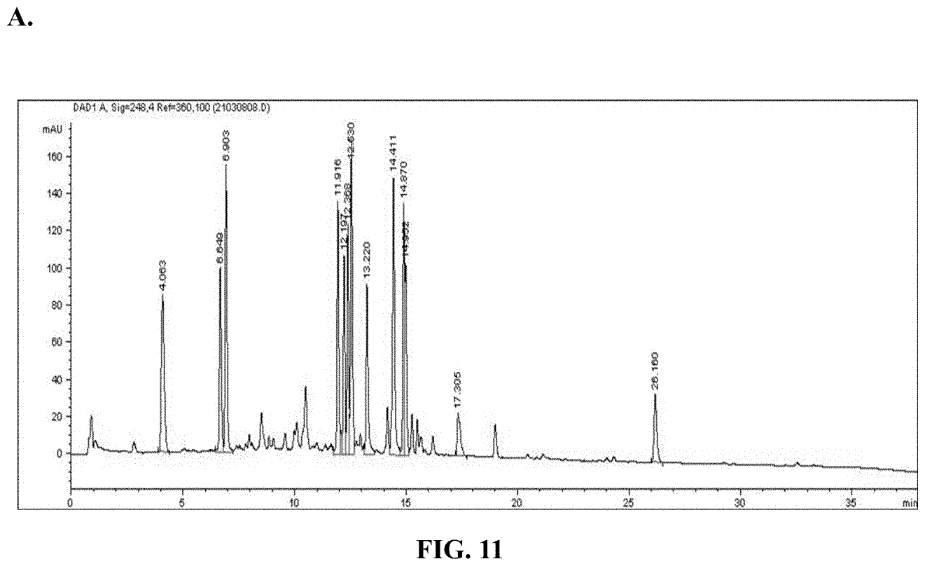

In the present disclosure, human lung cancer cells CL1-5 and CL1-0 were used as the model cell line to examine the effect of Ant-Ex or AE-F03 on ACE-2 expression. The effect of Ant-Ex on the expression of ACE-2 was first analyzed in CL1-5 cells. As shown in FIG. 1, ACE-2 was constitutively expressed in the CL1-5 cells. However, treatment of 50 μg/mL Ant-Ex for 24 hours significantly lower the ACE-2 protein expression as determined by western blot. In addition, 12.5-50 μg/mL of AE-F03 treatment also reduced the ACE-2 protein expression in both the CL1-5 cells, as determined by western blot (FIG. 10(A)).

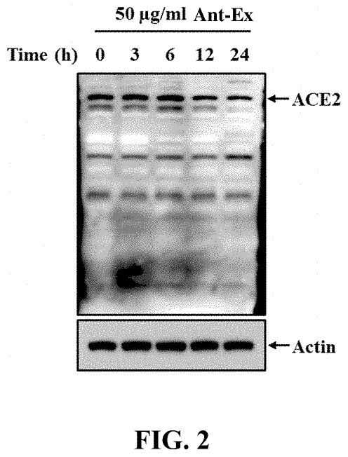

Subsequently, the effect of Ant-Ex on time-dependent reduction of ACE-2 was examined. The results showed that 50 μg/mL of Ant-Ex time-dependently reduced the ACE-2 protein expression, as determined by western blot (FIG. 2).

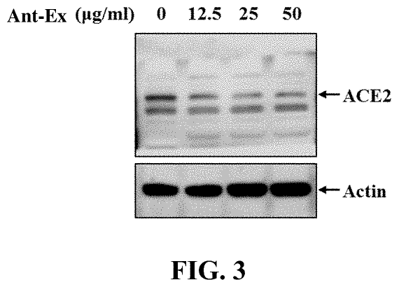

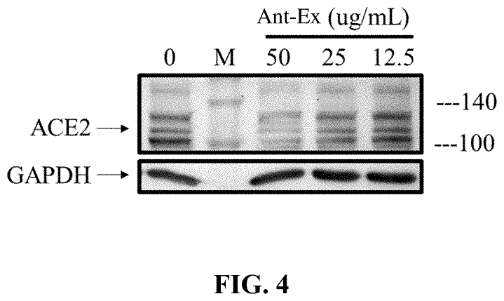

Next, the CL1-5 and CL1-0 cells were exposed to different concentrations of Ant-Ex for 24 hours, then the protein expression of ACE-2 was assessed using western blot. As shown in FIG. 3 and FIG. 4, Ant-Ex treatment reduced the ACE-2 protein expression in both the CL1-5 and CL1-0 cells in a dose-dependent manner.

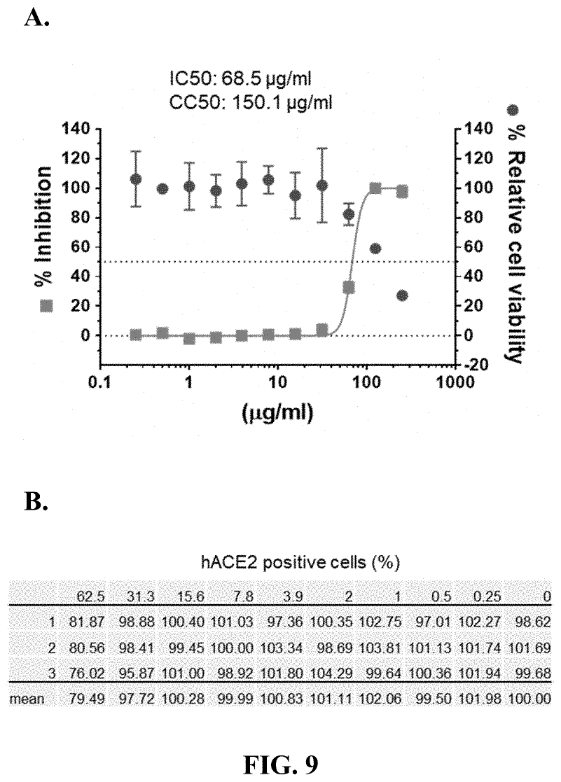

The AE-F03 comprising purified triterpenoid was assayed for its ACE-2 inhibitory potential in CL1-5 cells. The results showed that AE-F03 dose-dependently reduced the ACE-2 expression (FIG. 9(B)).

(ii) Ant-Ex Reduces ACE-2 mRNA Expression

Human lung cancer CL1-5 cells were incubated with 50 μg/mL Ant-Ex or vehicle for 1-3 hours, the ACE-2 mRNA expression levels were analyzed by RT-qPCR.

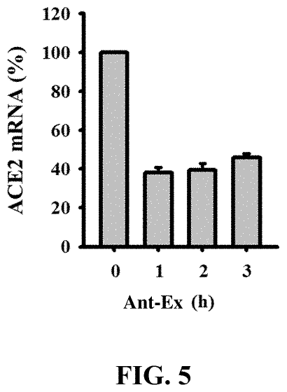

It is known that ACE-2 mRNA is mainly detected in small intestines, colon, duodenum, kidney, testis, and gallbladder. Normally, the ACE-2 mRNA expression level in the lung is low. However, under certain conditions, the upregulated ACE-2 expression in selected cells was observed. The effect of Ant-Ex on ACE-2 mRNA expression in CL1-5 cells was subsequently detected. The results showed that 50 μg/mL of Ant-Ex decreased the ACE-2 mRNA expression (FIG. 5). Notably, the ACE-2 mRNA expression was reduced to approximately 77% after 3 hours treatment (Tables 1-2 and FIG. 5), indicating that ACE-2 mRNA expression levels coincide with ACE-2 protein expression. As a result, it is found that with treatment of Ant-Ex to the lung cancer cells, both the mRNA and protein expression of ACE-2 are decreased.

TABLE 1

| | Test #1 | | Test #2 | |
|---|---|---|---|---|
| | ACE-2/ | % of control | ACE-2/ | % of control |
| | GAPDH | (ACE-2 mRNA) | GAPDH | (ACE-2 mRNA) |
| Control | 67.25 | 100 | 49.25 | 100 |
| 1 hour | | | | |
| Control | 29.29 | 100 | 26.56 | 100 |
| 3 hours | | | | |
| Ant-Ex | 16.22 | 24.12 | 12.69 | 25.77 |
| treatment | | | | |
| 1 hour | | | | |
| Ant-Ex | 22.75 | 77.67 | 16.63 | 62.26 |
| treatment | | | | |
| 3 hours | | | | |

TABLE 2

Test #3

| | | % of control |
|---|---|---|
| | ACE-2/GAPDH | (ACE-2 mRNA) |
| Control | 13.4 | 100 |
| 1 hour | | |
| Ant-Ex | 5.10 | 38.09 |
| treatment | | |
| 1 hours | | |
| Ant-Ex | 5.27 | 39.33 |
| treatment | | |
| 2 hour | | |
| Ant-Ex | 6.15 | 45.94 |
| treatment | | |
| 3 hours | | |

These results confirm that both Ant-Ex and AE-F03 were observed with inhibitory potential of ACE-2 in either mRNA or protein levels.

### Example 3: Ant-Ex and AE-F03 Inhibits Enzyme Activity of ACE-2

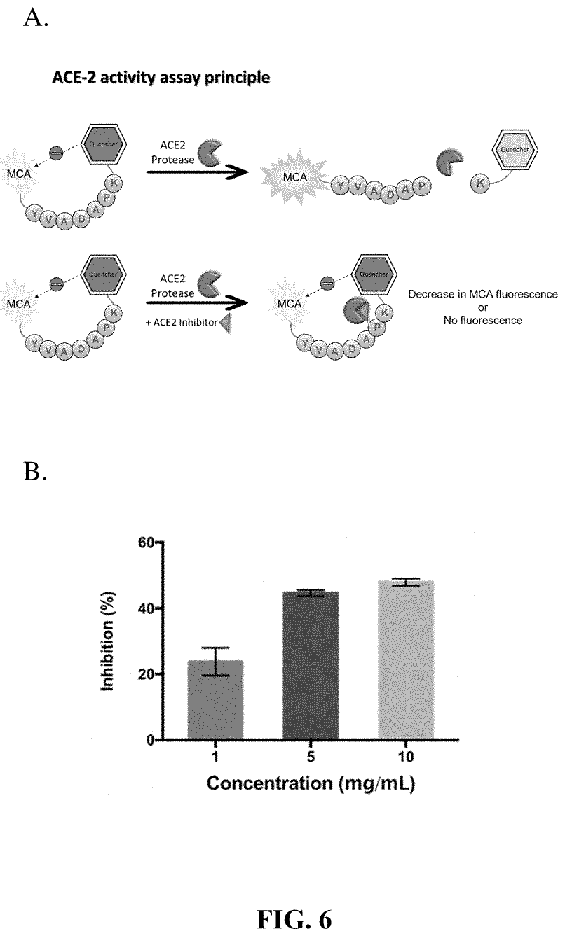

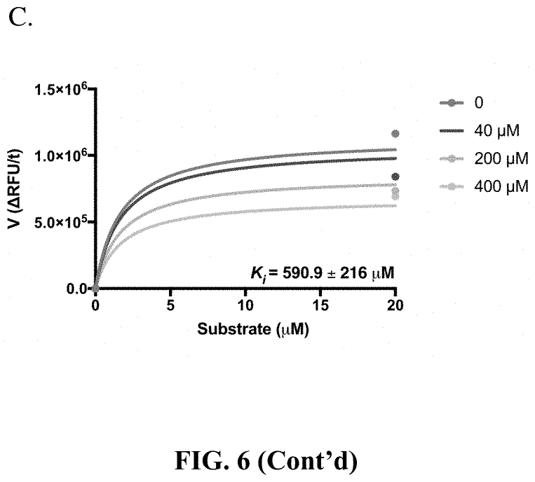

Various concentrations of Ant-Ex, AE-F03, Antcin A, Antcin B, Antcin C, Antcin H, Antcin K, dehydrosulphurenic acid (DSA) and dehydroeburicoic acid (DEA) were tested to see if they affect the enzyme activity of ACE-2. The assay utilizes the ability of an active ACE-2 to cleave a synthetic MCA based peptide substrate to release a free fluorophore from its quencher (FIG. 6(A)). The released MCA can be easily quantified using a fluorescence microplate reader.

Previous reports indicates that wild and solid-state culture A. cinnamomea methanol extract inhibit angiotensin-converting enzyme with an IC₅₀ values of 0.312 mg/mL, and 0.172 mg/mL, respectively. However, there is no information about the precise inhibitory mechanism and responsible compound. Concerns has emerged that angiotensin-converting enzyme inhibitors could theoretically increase the risk of SARS-CoV-2 infection owing to the role of ACE-2 as the viral binding site. However, it is known that angiotensin-converting enzyme inhibitors do not inhibit ACE-2 because angiotensin-converting enzyme and ACE-2 are entirely different enzymes.

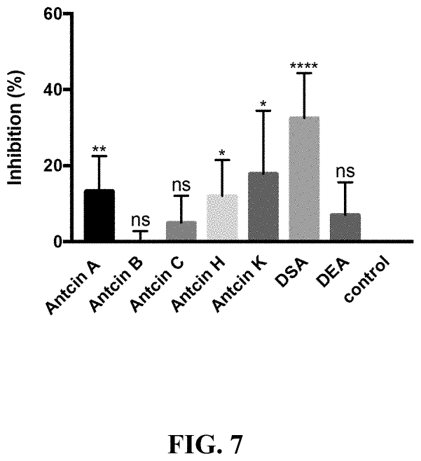

Ant-Ex were demonstrated to effectively inhibit ACE-2 enzyme activity (FIG. 6) by protein- and cell-based assays. In FIG. 10(B), AE-F03 was shown to have the ability of inhibiting ACE-2 by the enzyme activity assay on ACE-2 protein. Among the major constituents in Ant-Ex or AE-F03, except for Antcin B, all of these tested compounds had inhibition ability on ACE-2 enzymatic activity. Specifically, DSA and Antcin K were the strongest inhibitors with more than 30% inhibition on enzyme activity according to FIGS. 7 and 10(C).

### Example 4: Ant-Ex and AE-F03 Inhibit Binding of Spike Protein to ACE-2

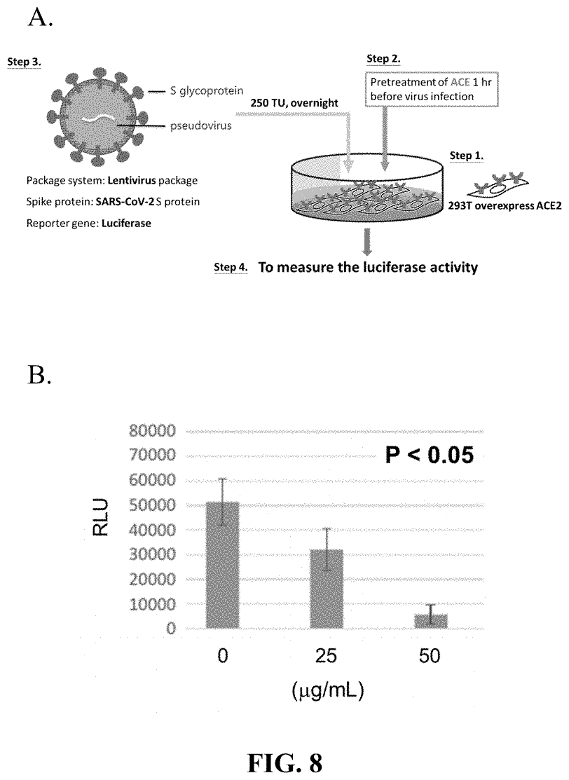

To confirm whether the reduction in the expression and the enzyme activity of ACE-2 is associated with inhibition of virus binding, pseudovirus with SARS-CoV-2 S protein and carrying a luciferase reporter gene and cells which overexpress ACE-2 protein on the cell surface were applied (FIG. 8(A)). The assay system was performed to see if ACE inhibit the interaction between SARS-CoV-2 Spike protein (S protein) and ACE-2 on the cell surface.

In addition, Vero-E6 cells were pretreated with the Ant-Ex (FIG. 9(A)) or AE-F03 (FIG. 10(D)) at the indicated concentration for 1 hour at 37° C. and then were adsorbed with SARS-CoV-2 at 100 PFU (MOI=0.01) for 1 hour at 37° C. The virus was removed and cells were added with fresh medium with the compound at the indicated concentration for 1 day incubation. Cells were fixed and immunostained with anti-SARS-CoV-2 N protein antibody plus anti-human IgG-488. For cell viability test, the Vero-E6 cells were treated with the ACE at the indicated concentration for 1 day at 37° C. The cell viability was determined by Cell Counting Kit-8 (CCK-8). The fluorescent signal was quantified by high-content imaging and the infection rate of no compound treatment was set as 100%. The 50% inhibition concentration (IC50) and 50% cytotoxic concentration (CC50) were calculated by Prism software.

Owing to the result shown in FIG. 8(B), Ant-Ex significantly inhibited the interaction between S protein and ACE-2. According to the results shown in FIGS. 9(A) and 10(D), Ant-Ex indeed significantly inhibited SARS-CoV-2 to infect Vero-E6 cells by IC50 of 68.5 μg/mL (FIG. 9(A)) while AE-F03 inhibited SARS-CoV-2 to infect Vero-E6 cells by IC50 of 82.92 μg/mL (FIG. 10(D)). These demonstration clearly showed that the Ant-Ex, AE-F03 or constituents thereof can be administered as the important prophylactic agent or drugs to prevent a subject from SARS-CoV-2 infection and other virus infection which infect a subject through the similar mechanism of binding to ACE-2 receptor.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method of treating, reducing the risk of, preventing, or alleviating angiotensin converting enzyme 2 (ACE-2) associated state in a subject, comprising administering to the subject a therapeutically effective amount of Antrodia cinnamomea extract (Ant-Ex) or fraction 3 thereof (AE-F03).

2. The method of claim 1, wherein said subject is mammalian.

3. The method of claim 2, wherein said subject is human.

4. The method of claim 1, wherein said ACE-2 associated state is a blood pressure related disease or disorder.

5. The method of claim 1, wherein said ACE-2 associated state is selected from the group consisting of: chronic heart failure, left ventricular hypertrophy, acute heart failure, and cardiomyopathy.

6. The method of claim 1, wherein said ACE-2 associated state is congestive heart failure, arterial hypertension or myocardial infarction.

7. The method of claim 1, wherein said ACE-2 associated state is a cell proliferation disorder.

8. The method of claim 7, wherein the cell proliferation disorder is a smooth cell proliferation disorder.

9. The method of claim 8, wherein said smooth cell proliferation disorder is vascular stenosis.

10. The method of claim 1, wherein said ACE-2 associated state is a kidney disease or disorder.

11. The method of claim 1, wherein said ACE-2 associated state is a kinetensin associated disorder.

12. The method of claim 11, wherein said kinetensin associated disorder is selected from the group consisting of: abnormal vascular permeability, local and systemic allergic reactions, eczema, asthma, and anaphylactic shock.

13. The method of claim 1, wherein said ACE-2 associated state is inflammation.

14. The method of claim 13, wherein said inflammation is selected from the group consisting of: systemic inflammatory response syndromes (SIRS), polytrauma, inflammatory bowel disease, acute and chronic pain, bone destruction in rheumatoid and osteo arthritis, periodontal disease, dysmeorrhea, premature labor, brain edema following focal injury, diffuse axonal injury, allergic disorders, wound healing, and scar formation.

15. The method of claim 1, wherein said ACE-2 associated state is virus infection.

16. The method of claim 15, wherein said virus infection comprises corona virus infection.

17. The method of claim 16, wherein said corona virus infection comprises severe acute respiratory syndrome coronavirus (SARS-CoV) infection, Middle East respiratory syndrome coronavirus (MERS-CoV) infection or severe acute respiratory syndrome coronavirus 2 (SARS-CoV-2) infection.

18. The method of claim 1, wherein said Antrodia cinnamomea extract (Ant-Ex) is obtained by:
extracting dried fruiting bodies of Antrodia cinnamomea via 95% (v/v) ethanol.

19. The method of claim 1, wherein said Antrodia cinnamomea extract comprises a fraction 3 thereof (AE-F03).

20. A method of manufacturing a drug or food supplement useful for treating, reducing the risk of, preventing, or alleviating angiotensin converting enzyme 2 (ACE-2) associated state, wherein said method comprises adding Antrodia cinnamomea extract (Ant-Ex) or fraction 3 thereof (AE-F03) to a drug or a food.

21. A method of reducing angiotensin converting enzyme 2 (ACE-2) expression in a subject in need thereof comprising administering to the subject Antrodia cinnamomea extract (Ant-Ex) or fraction 3 thereof (AE-F03).
